(19) United States
(12) Patent Application Publication
(10) Pub. No.: US 2006/0183043 A1
(43) Pub. Date: Aug. 17, 2006

(54) Electrophotographic photoreceptor containing pyridine-substituted asymmetric naphthalenetetracarboxylic acid diimide derivatives and electrophotographic imaging apparatus

(75) Inventors: Seung-ju Kim, Suwon-si (KR); Beom-jun Kim, Yongin-si (KR); Hwan-koo Lee, Suwon-si (KR); Ji-Young Lee, Suwon-si (KR); Saburo Yokota, Suwon-si (KR); Moto Makino, Suwon-si (KR)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: Samsung Electronics Co., Ltd.

(21) Appl. No.: 11/352,381

(22) Filed: Feb. 13, 2006

(30) Foreign Application Priority Data
Feb. 14, 2005 (KR) 10-2005-0011910

(51) Int. Cl. G03G 5/05; C07D 471/02

(52) U.S. Cl. 430/78; 546/66

(57) ABSTRACT

An electrophotographic photoreceptor includes a pyridine-substituted asymmetric naphthalenetetracarboxylic acid diimide derivative. The pyridine-substituted asymmetric naphthalenetetracarboxylic acid diimide derivative has increased solubility in organic solvents, improved compatibility with binder resins and enhanced resistance for nitrogen oxides. The electrophotographic photoreceptor containing the asymmetric naphthalenetetracarboxylic acid diimide compound according to the present invention can maintain a constant surface potential and durability after being repeatedly used for an extended time. The electrophotographic photoreceptor according to the present invention can provide a high image quality for an extended time. The invention is also directed to an electrophotographic imaging apparatus, an electrophotographic cartridge and to novel pyridine-substituted asymmetric naphthalenetetracarboxylic acid diimide derivatives.

### CROSS-REFERENCE TO RELATED PATENT APPLICATION

This application claims the benefit of Korean Patent Application No. 10-2005-0011910, filed on Feb. 14, 2005, in the Korean Intellectual Property Office, the disclosure of which is hereby incorporated by reference in its entirety.

### BACKGROUND OF THE INVENTION

1. Field of the Invention

The present invention relates to an electrophotographic photoreceptor containing pyridine-substituted asymmetric naphthalenetetracarboxylic acid diimide derivatives and an electrophotographic imaging apparatus. More particularly, the invention is directed to an electrophotographic photoreceptor containing naphthalenetetracarboxylic acid diimide derivatives having good solubility to an organic solvent and high compatibility with a polymeric binder resin. The invention is also directed to an electrophotographic imaging apparatus including the electrophotographic photoreceptor.

2. Description of the Related Art

An electrophotographic photoreceptor is used in electrophotography applied to laser printers, photocopiers, CRT printers, facsimile machines, LED printers, liquid crystal printers, and laser electrophotos. The electrophotographic photoreceptor comprises a photosensitive layer formed on an electrically conductive substrate. The substrate can be in the form of a plate, a disk, a sheet, a belt, a drum, or other structure. In electrophotography, an image is formed using an electrophotographic photoreceptor. First, a surface of the photosensitive layer is electrostatically charged uniformly, and then the charged surface is exposed to a pattern of light to form an image. The light exposure selectively dissipates the charge in the exposed regions where the light strikes the surface, thereby forming a pattern of charged and uncharged regions, which is referred to as a latent image. Then, a wet or dry toner is applied in the vicinity of the latent image, and toner droplets or particles deposit in either the charged or uncharged regions to form a toner image on the surface of the photosensitive layer. The resulting toner image can be transferred and fixed to a suitable ultimate or intermediate receiving surface, such as paper, or the photosensitive layer can function as the ultimate receptor for receiving the image. The residual toner is then cleaned and residual charges are erased from the electrophotographic photoreceptor. Thus, the electrophotographic photoreceptor can be repeatedly used for long periods.

Electrophotographic photoreceptors are generally categorized into two types. The first is a laminated type having a laminated structure including a charge generating layer comprising a binder resin and a charge generating material (CGM), and a charge transporting layer comprising a binder resin and a hole transporting material (HTM). In general, the laminated type electrophotographic photoreceptor is used in the fabrication of a negative (−) type electrophotographic photoreceptor. The other type is a single layered type in which a binder resin, a CGM, an HTM, and an electron transporting material (ETM) are contained in a single layer. In general, the single layered type photoreceptor is used in the fabrication of a positive (+) type electrophotographic photoreceptor.

The (+) type single layered electrophotographic photoreceptor is advantageous in that it generates only a small amount of ozone harmful to humans and since it has a single photosensitive layer, its production costs are low. The most essential material among the materials of the (+) type single layered electrophotographic photoreceptor is the ETM. Since the hole transporting ability of the HTM is at least a hundred times greater than the electron transporting ability of the commonly used ETM, the performance of the single layered electrophotographic photoreceptor is dependent upon the electron transporting ability of the ETM.

The electron transporting ability of the ETM is greatly affected by its solubility in an organic solvent and compatibility with a polymer binder resin. The conventional ETM includes a dicyanofluorenone derivative having Formula (i) below, a diphenoquinone derivative having Formula (ii) below, a naphthalenetetracarboxylic acid diimide derivative having Formula (iii) as disclosed in U.S. Pat. Nos. 4,992,349 and 4,442,193, and an o-substituted naphthalenetetracarboxylic acid diimide derivative having Formula iv as disclosed in U.S. Pat. No. 6,127,076).

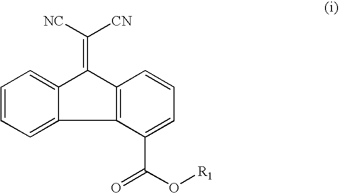

wherein

R₁ is a substituted or unsubstituted alkyl group or an aryl group,

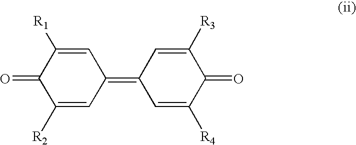

wherein

each of R₁, R₂, R₃, and R₄ is independently a substituted or unsubstituted alkyl group, a cycloalkyl group, an aryl group, or an alkoxy group,

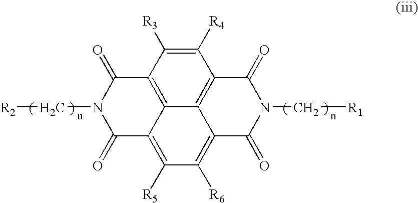

wherein

R₁ and R₂ are independently a substituted or unsubstituted aryl group, a sulfonyl group, a sulfone group,

R₃, R₄, R₅, and R₆ are independently a hydrogen atom, a C₁₋₄ alkyl group, a C₁₋₄ alkoxy group, or a halogen atom, and

n is 0-3,

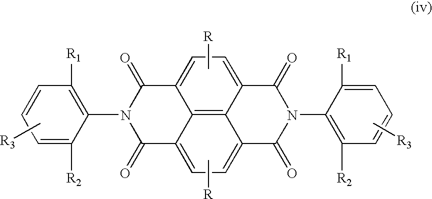

wherein

R is a hydrogen atom, an alkyl group, an alkoxyl group, or a halogen atom,

R₁ and R₂ are different from each other and each a substituted or unsubstituted alkyl group, an alkoxyl group, or an aryl group,

R₃ is a hydrogen atom, a substituted or unsubstituted alkyl group, an alkoxyl group, or an aryl group.

The dicyanofluorenone derivative of Formula (i) and the diphenoquinone derivative of Formula (ii) have low solubility in organic solvents and low inherent electron transporting ability. Thus, electrophotographic photoreceptors manufactured using the derivative (i) or (ii) as the ETM have disadvantages such as a remarkably reduced charge potential and an increased exposure potential after repeated charging exposures.

The naphthalenetetracarboxylic acid diimide derivatives of Formulae (iii) and (iv) are known to have high electron transporting ability. However, these derivatives of Formulae (iii) and (iv) have low solubility in organic solvents and low compatibility with polymer binder resins. Electrophotographic photoreceptors manufactured using these derivatives have surfaces of the photosensitive layers that may crystallize, thus adversely affecting the electrostatic properties of the photoreceptors.

Thus, electrophotographic photoreceptors, especially single layered type electrophotographic photoreceptors, manufactured using the conventional ETMs have a remarkably reduced charge potential and an increased exposure potential after repeated use. In general, surface charges of electrophotographic photoreceptors must be maintained at a predetermined potential. Due to the decrease in the charge potential and the increases in the exposure potential, image qualities may be deteriorated.

### SUMMARY OF THE INVENTION

The present invention provides an electrophotographic photoreceptor including new naphthalenetetracarboxylic acid diimide derivatives having an effective solubility in organic solvents and an effective compatibility with polymer binder resins, thus providing effective electron transporting ability.

The present invention also provides an electrophotographic imaging apparatus and an electrophotographic cartridge employing the electrophotographic photoreceptor.

The present invention also provides new naphthalenetetracarboxylic acid diimide derivatives.

According to an aspect of the present invention, an electrophotographic photoreceptor includes an electrically conductive substrate, and a photosensitive layer formed on the electrically conductive substrate, wherein the photosensitive layer comprises a pyridine-substituted asymmetric naphthalenetetracarboxylic acid diimide derivative represented by Formula (1): 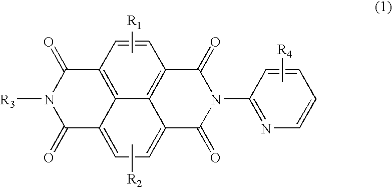
wherein R₁, R₂ R₃ and R₄ are independently selected from the group consisting of a substituted or unsubstituted C₁-C₂₀ alkyl group, a substituted or unsubstituted C₁-C₂₀ alkoxy group, a substituted or unsubstituted C₆-C₃₀ aryl group, a substituted or unsubstituted C₇-C₃₀ aralkyl group, a substituted or unsubstituted C₃-C₃₀ heterocyclic group, and a halogen atom.

According to another aspect of the present invention, an electrophotographic imaging apparatus includes an electrophotographic photoreceptor, wherein the electrophotographic photoreceptor includes an electrically conductive substrate, and a photosensitive layer formed on the electrically conductive substrate, wherein the photosensitive layer comprises a pyridine-substituted asymmetric naphthalenetetracarboxylic acid diimide derivative represented by Formula (1).

According to still another aspect of the present invention, there is provided an electrophotographic imaging apparatus including an electrophotographic photoreceptor unit including an electrically conductive substrate, and a photosensitive layer formed on the electrically conductive substrate, wherein the photosensitive layer comprises a pyridine-substituted asymmetric naphthalenetetracarboxylic acid diimide derivative represented by Formula (1), a charging device that charges the photoreceptor unit, an imagewise light irradiating device that irradiates the charged photoreceptor unit with imagewise light to form an electrostatic latent image on the photoreceptor unit, a developing unit that develops the electrostatic latent image with a toner to form a toner image on the photoreceptor unit, and a transfer unit that transfers the toner image onto a receiving material.

According to yet another aspect of the present invention, an electrophotographic cartridge including an electrophotographic photoreceptor comprises an electrically conductive substrate, and a photosensitive layer formed on the electrically conductive substrate, wherein the photosensitive layer comprises a pyridine-substituted asymmetric naphthalenetetracarboxylic acid diimide derivative represented by Formula (1), and at least one selected from the group consisting of a charging device that charges the electrophotographic photoreceptor, a developing device that develops an electrostatic latent image formed on the electrophotographic photoreceptor, and a cleaning device that cleans a surface of the electrophotographic photoreceptor, the electrophotographic cartridge being attachable to or detachable from the imaging apparatus.

In the electrophotographic imaging apparatus, an intermediate layer may further be provided between the electrically conductive substrate and the photosensitive layer.

According to a further aspect of the present invention, a pyridine-substituted asymmetric naphthalenetetracarboxylic acid diimide derivative is represented by Formula (1).

These and other aspects of the invention will become apparent from the following detailed description of the invention, which taken in conjunction with the annexed drawings, disclose embodiments of the invention.

### BRIEF DESCRIPTION OF THE DRAWINGS

The above and other features and advantages of the present invention will become more apparent by describing in detail exemplary embodiments thereof with reference to the attached drawings in which:

FIG. 1 is a schematic view of an imaging apparatus, an electrophotographic drum, and an electrophotographic cartridge in accordance with an embodiment of the present invention;

FIG. 2 is a ¹H-NMR spectrum of naphthalenetetracarboxylic acid diimide derivative according to Preparation Example 1 of the present invention (Compound 1);

FIG. 3 is a ¹H-NMR spectrum of naphthalenetetracarboxylic acid diimide derivative according to Preparation Example 2 of the present invention (Compound 4); and

FIG. 4 is a ¹H-NMR spectrum of naphthalenetetracarboxylic acid diimide derivative according to Preparation Example 3 of the present invention (Compound 5).

### DETAILED DESCRIPTION OF THE INVENTION

An electrophotographic photoreceptor according to the present invention and an electrophotographic imaging apparatus employing the same will now be described in detail.

The pyridine-substituted naphthalenetetracarboxylic acid diimide derivative having Formula 1 according to the present invention has an asymmetric structure, and has improved solubility in organic solvents and an excellent compatibility with polymer binder resins. In addition, by introducing a pyridine group having a high electron affinity to the naphthalenetetracarboxylic acid diimide derivative, the electron transporting ability of the naphthalenetetracarboxylic acid diimide derivative can be further enhanced.

In the electrophotographic image forming apparatus, nitrogen oxides (NOx) are generated during corona charging, which shorten the life span of the electrophotographic photoconductive material. However, pyridine may serve as an acid acceptor and thus, it is possible to inhibit the effects of the NOx and increase the life span of the electrophotographic photoconductive material by using naphthalenetetracarboxylic acid diimide derivatives that include a pyridine structure to effectively remove the nitrogen oxides.

Accordingly, the use of the pyridine-substituted asymmetric diimide derivative according to the present invention as an electron transporting material (ETM) represented by Formula 1 provides for an electrophotographic photoreceptor having effective electrostatic property and durability. 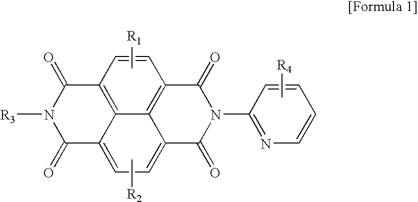
wherein R₁, R₂ R₃ and R₄ are independently selected from the group consisting of a substituted or unsubstituted C₁-C₂₀ alkyl group, a substituted or unsubstituted C₁-C₂₀ alkoxy group, a substituted or unsubstituted C₆-C₃₀ aryl group, a substituted or unsubstituted C₇-C₃₀ aralkyl group, a substituted or unsubstituted C₃-C₃₀ heterocyclic group, and a halogen atom.

The halogen atom is preferably fluorine, chlorine, bromine or iodine.

The alkyl group is a linear or branched C₁-C₂₀ alkyl group, preferably a linear or branched C₁-C₁₂ alkyl group. Examples of the alkyl group include methyl, ethyl, n-propyl, isopropyl, n-butyl, isobutyl, sec-butyl, tert-butyl, pentyl, hexyl, 1,2-dimethyl-propyl, and 2-ethylhexyl. The alkyl group may be substituted with a halogen atom, an alkyl group, an aryl group, an alkoxy group, a nitro group, a hydroxy group, or a sulfonic acid group.

The alkoxy group is a linear or branched C₁-C₂₀ alkoxy group, and preferably a linear or branched C₁-C₁₂ alkoxy group. Examples of the alkoxy group include methoxy, ethoxy, propoxy, and the like. The alkoxy group may be substituted with a halogen atom, an alkyl group, an aryl group, an alkoxy group, a nitro group, a hydroxyl group, or a sulfonic acid group.

The aralkyl group is a linear or branched C₇-C₃₀ aralkyl group, and preferably a linear or branched C₇-C₁₅ aralkyl group. Examples of the aralkyl group include benzyl, methylbenzyl, phenylethyl, naphthylmethyl, and naphthylethyl. The aralkyl group may be substituted with a halogen atom, an alkyl group, an aryl group, an alkoxy group, a nitro group, a hydroxyl group, or a sulfonic acid group.

The aryl group is a C₆-C₃₀ aromatic ring. Examples of the aryl group include phenyl, tolyl, xylyl, biphenyl, o-terphenyl, naphtyl, anthracenyl, phenanthrenyl, and the like. The aryl group may be a substituted or unsubstituted aryl group and may be substituted with an alkyl group, an alkoxy group, a nitro group, a hydroxyl group, a sulfonic acid group, or a halogen atom.

The heterocyclic group is a substituted or unsubstituted C₃-C₃₀ heterocyclic ring group which includes any monocyclic or polycyclic (e.g., bicyclic, tricyclic, etc.) ring compound having at least a heteroatom (e.g., O, S, N, P, B, Si, etc.) in the ring. Examples of the heterocyclic group include pyridyl, pyrimidinyl, indolyl, quinolinyl, isoquinolinyl, oxazolyl, and imidazolyl.

Specific examples of the pyridine-substituted asymmetric naphthalenetetracarboxylic acid diimide derivatives having Formula (1) include the following Compounds 1-5:

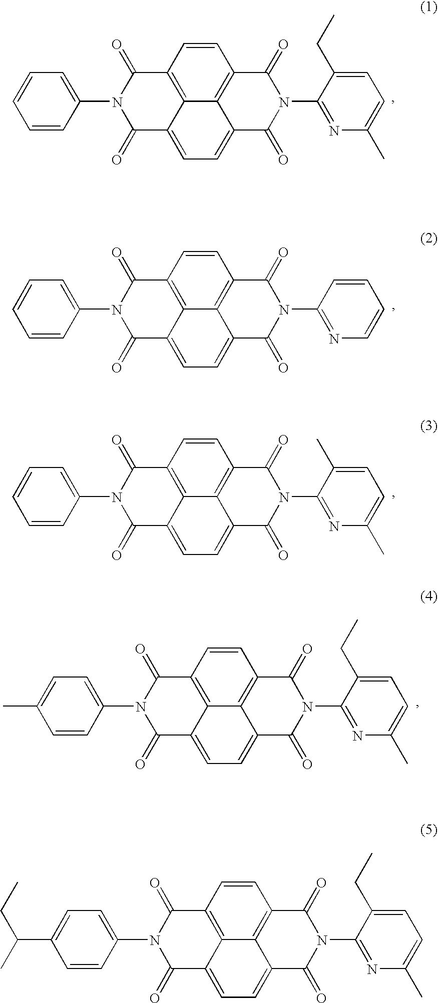

As evident from structures of Compounds (1) through (5), the naphthalenetetracarboxylic acid diimide derivative according to the present invention has a pyridine-substituted asymmetric structure. The term “asymmetric” as used herein includes, for example, the following case; i) a substituted or unsubstituted pyridine group is bonded to one nitrogen atom of an imide bond of the naphthalenetetracarboxylic acid diimide derivative while a substituted or unsubstituted alkyl group, aryl group or aralkyl group and the like other than a pyridine group is bonded to the other nitrogen atom of the other imide bond, ii) two pyridine groups are bonded to each nitrogen atom of two imide bonds of the naphthalenetetracarboxylic acid diimide derivative, respectively, wherein the two pyridine groups have different substituents on their respective pyridine rings. Because of such an asymmetric structure, the diimide derivative of the present invention has improved solubility in organic solvents and excellent compatibility with polymer binder resins. Accordingly, the diimide derivative according to the present invention exhibits noticeably improved electron transporting ability compared to the prior compounds. In addition, the diimide derivative according to the present invention provides enhanced durability of the electrophotographic photoreceptor by introducing a pyridine group having a high electron affinity thereto.

Next, a method of preparing the pyridine-substituted asymmetric naphthalenetetracarboxylic acid diimide derivative according to the present invention will be described.

The naphthalenetetracarboxylic acid diimide derivative according to the present invention is prepared by reacting a naphthalenetetracarboxylic acid dianhydride having Formula (2) with an amine compound having Formula (3) and an amine compound having Formula (4): 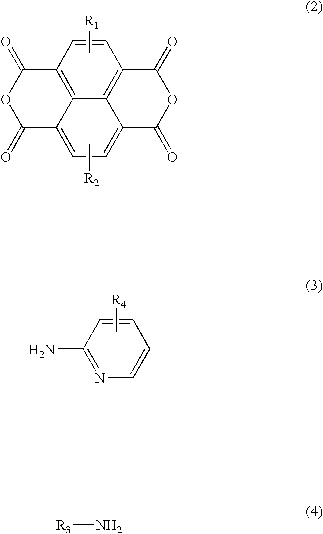
wherein R₁, R₂, R₃, and R₄ are as defined above.

In the reaction, a polar organic solvent, for example, dimethylformamide (DMF), dimethylacetamide (DMAc), hexamethylphosphoamide (HMPA), or N-methy-2-pyrrolidone (NMP), may be used. The reaction temperature may be set in the range of 20° C. below than the boiling point of the solvent to the boiling point of the solvent, and preferably, in the range of 10° C. below than the boiling point of the solvent to the boiling point of the solvent.

Generally, the reaction may be carried out in the following manner. First, the naphthalenetetracarboxylic acid dianhydride compound represented by formula (2) is dissolved in a polar organic solvent such as DMF, DMAc, HMPA, or NMP, and then the compounds having Formulas (3) and (4) are added dropwise to the resulting solution. Then, the mixture is refluxed for 3 to 24 hours, preferably 3 to 10 hours, to obtain the pyridine-substituted asymmetric naphthalenetetracarboxylic diimide derivative represented by Formula (1). In the reaction, the naphthalenetetracarboxylic acid dianhydride of Formula (2), the pyridine compound having Formula (3), and the amine compound having Formula (4) may be used in a molar ratio of 1:1:1 to 1:2:2. In the reaction, when the pyridine compound having Formula (3) or the amine compound having Formula (4) are bonded to both nitrogen atoms in imide bonds of the compound having Formula (2), a symmetric naphthalenetetracarboxylic acid diimide derivative is obtained. The symmetric naphthalenetetracarboxylic acid diimide derivative has much lower solubility in organic solvents than the pyridine-substituted asymmetric naphthalenetetracarboxylic acid diimide derivative according to the present invention. Therefore, the pyridine-substituted asymmetric naphthalenetetracarboxylic acid diimide derivative according to the present invention can be separated using the difference in the solubility in organic solvents.

An electrophotographic imaging apparatus and an electrophotographic cartridge employing the electrophotographic photoreceptor according to the present invention comprising the pyridine-substituted asymmetric naphthalenetetracarboxylic acid diimide derivative having Formula (1) will now be described.

FIG. 1 schematically illustrates an image forming apparatus 30 including an electrophotographic photoreceptor drum 28, 29 and an electrophotographic cartridge 21 according to an embodiment of the present invention. The electrophotographic cartridge 21 typically includes an electrophotographic photoreceptor 29, one or more charging devices 25 for charging the electrophotographic photoreceptor 29, a developing device 24 for developing an electrostatic latent image formed on the electrophotographic photoreceptor 29, and a cleaning device 26 for cleaning a surface of the electrophotographic photoreceptor 29 to remove residual toner. The electrophotographic cartridge 21 can be attached to and detached from the image forming apparatus 30.

The electrophotographic photoreceptor drum 28, 29 of the image forming apparatus 30 can generally be attached to and detached from the image forming apparatus 30 and includes the drum 28 on which the electrophotographic photoreceptor 29 is placed.

Generally, the image forming apparatus 30 includes a photosensitive unit (for example, the drum 28 and the electrophotographic photoreceptor 29); the charging device 25 for charging the photoreceptor unit; an image-forming light device 22 for irradiating light onto the charged photoreceptor unit to form an electrostatic latent image on the photoreceptor unit. The developing unit 24 is included for developing the electrostatic latent image with a toner to form a toner image on the photoreceptor unit. A transfer device 27 is provided for transferring the toner image onto a receiving material, such as paper P. The photoreceptor unit 28 includes the electrophotographic photoreceptor 29, as described below. The charging device 25 may be supplied with a voltage to charge the electrophotographic photoreceptor 29. The image forming apparatus 30 may also include a pre-exposure unit 23 to erase residual charges on the surface of the electrophotographic photoreceptor 29 to prepare for a next cycle.

The electrophotographic photoreceptor including the pyridine-substituted asymmetric naphthalenetetracarboxylic acid diimide derivative naphthalenetetracarboxylic diimide derivative having Formula (1) according to an embodiment of the present invention may be incorporated into electrophotographic imaging apparatuses such as laser printers, photocopiers, or facsimiles.

The electrophotographic photoreceptor according to the present invention including the pyridine-substituted asymmetric naphthalenetetracarboxylic acid diimide derivative represented by Formula (1) employed in the electrophotographic imaging apparatus shown in FIG. 1 will now be described.

The electrophotographic photoreceptor comprises a photosensitive layer formed on an electrically conductive substrate. The electrically conductive substrate may be composed of metal, an electrically conductive polymer, or other materials and is produced in the form of a plate, a disk, a sheet, a belt, or a drum. Examples of the metal include aluminum and stainless steel. Examples of the electrically conductive polymer include polyester resin, polycarbonate resin, polyamide resin, polyimide resin, mixtures and copolymers thereof in which an electrically conductive material, such as electrically conductive carbon, tin oxide, indium oxide, is dispersed.

The photosensitive layer may be a laminated type where a charge generating layer and a charge transporting layer are separately formed, or a single layered type where a layer acts as both a charge generating layer and a charge transporting layer.

The naphthalenetetracarboxylic acid diimide derivative of Formula (1) according to the present invention acts as a charge transporting material, and preferably, as an ETM. In the laminated type photosensitive layer, the naphthalenetetracarboxylic acid diimide derivative of Formula (1) is contained in the charge transporting layer. In the single layered type photosensitive layer, the derivative of Formula (1) is naturally contained in a single layer together with a charge generating material (CGM).

Examples of the CGM used in the photosensitive layer include organic materials such as phthalocyanine pigments, azo pigments, quinone pigments, perylene pigments, indigo pigments, bisbenzoimidazole pigments, quinacridone pigments, azulenium dyes, squarylium dyes, pyrylium dyes, triarylmethane dyes, and cyanine dyes, and inorganic materials such as amorphous silicon, amorphous selenium, trigonal selenium, tellurium, selenium-tellurium alloy, cadmium sulfide, antimony sulfide, and zinc sulfide. The CGM is not limited to the materials listed herein, and may be used alone or in a combination of two or more.

In the laminated type photoreceptor, the CGM is dispersed in a solvent with a binder resin and then the dispersion is coated on the electrically conductive substrate by a dip coating, a ring coating, a roll coating, or a spray coating method to form the charge generating layer. The thickness of the charge generating layer is generally about 0.1-1 μm. When the thickness is less than 0.1 μm, the sensitivity is insufficient, and when the thickness is greater than 1 μm, the charging ability and the sensitivity are lowered.

A charge transport layer containing the naphthalenetetracarboxylic acid diimide derivative of Formula (1) is formed on the charge generating layer of the laminated type photosensitive layer, but the charge generating layer may be formed on the charge transport layer in reverse order. When forming the charge transport layer, the naphthalenetetracarboxylic acid diimide derivative of Formula (1) and the binder resin are dissolved in a solvent and the resulting solution is coated on the charge generating layer. Examples of the coating method include a dip coating, a ring coating, a roll coating, and a spray coating method, similar to the methods used to form the charge generating layer. The thickness of the charge transport layer is generally about 5-50 μm. When the thickness is less than 5 μm, the charging ability becomes poor, and when the thickness is greater than 50 μm, the response rate is reduced and the image quality is deteriorated.

When preparing the single layered photoreceptor, the CGM is dispersed in a solvent together with the binder resin and the pyridine-substituted asymmetric naphthalenetetracarboxylic acid diimide derivative of Formula (1) as the ETM and the resulting dispersion is coated on the electrically conductive substrate to obtain the photosensitive layer. The thickness of the photosensitive layer is generally about 5-50 μm. When the thickness of the single layered photosensitive layer is less than 5 μm, the charging capability and sensitivity are lowered. When the thickness of the single layered photosensitive layer is greater than 50 μm, a residual potential may increase or the response speed may decrease. The pyridine-substituted asymmetric naphthalenetetracarboxylic acid diimide derivative of Formula (1) may be used together with other ETM and/or HTM. Especially, in the single layered photoreceptor, it is preferable to use the naphthalenetetracarboxylic acid diimide derivatives of Formula (1) together with the HTM.

Examples of the HTM that may be used with the naphthalenetetracarboxylic acid diimide derivatives of Formula (1) in the photosensitive layer include nitrogen containing cyclic compounds or condensed polycyclic compounds such as pyrene compounds, carbazole compounds, hydrazone compounds, oxazole compounds, oxadiazole compounds, pyrazoline compounds, arylamine compounds, arylmethane compounds, benzidine compounds, thiazole compounds or styryl compounds. Also, high molecular weight compounds having functional groups of the above compounds on a backbone or side chain may be used.

Examples of other ETM that may be used with the naphthalenetetracarboxylic acid diimide derivatives of Formula (1) in the photosensitive layer include, but are not limited to, electron attracting low-molecular weight compounds such as benzoquinone compounds, cyanoethylene compounds, cyanoquinodimethane compounds, fluorenone compounds, xanthone compounds, phenanthraquinone compounds, anhydrous phthalic acid compounds, thiopyrane compounds, or diphenoquinone compounds. Electron transporting polymer compounds or pigments having n-type semiconductor characteristic may also be used.

The ETM or the HTM that may be used with the naphthalenetetracarboxylic acid diimide derivatives of Formula (1) in the electrophotographic photoreceptor are not limited to the materials listed herein, and the foregoing materials may be used alone or in combination of two or more.

Examples of solvents used in preparing a coating composition for forming the photosensitive layer include organic solvents such as alcohols, ketones, amides, ethers, esters, sulfones, aromatics, halogenated aliphatic hydrocarbons, and the like. The coating method of the coating composition may be a dip coating method, but a ring coating, a roll coating, a spray coating method, or the like may be also used.

Examples of the binder resin used in the formation of the photosensitive layer include, but are not limited to, polycarbonate, polyester, methacryl resin, acrylic resin, polyvinyl chloride, polyvinylidene chloride, polystyrene, polyvinyl acetate, silicon resin, silicon-alkyd resin, styrene-alkyd resin, poly-N-vinylcarbazole, phenoxy resin, epoxy resin, polyvinyl butyral, polyvinyl acetal, polyvinyl formal, polysulfone, polyvinyl alcohol, ethyl cellulose, phenolic resin, polyamide, carboxy-methyl cellulose and polyurethane. These polymers may be used alone or in a combination of two or more.

The amount of the CTM including the ETM and the HTM in the photoconductive layer may be in the range of 10-60% by weight based on total weight of the photosensitive layer. If the amount is less than 10% by weight, the sensitivity is insufficient due to low charge transporting ability, thereby resulting in an increased residual potential. If the amount is more than 60% by weight, the amount of the resin in the photosensitive layer is reduced, thereby reducing mechanical strength.

In an embodiment of the present invention, an electroconductive layer may further be formed between the substrate and the photosensitive layer. The electroconductive layer is obtained by dispersing an electroconductive powder such as carbon black, graphite, metal powder or metal oxide powder in a solvent and then applying the resulting dispersion on the substrate and drying it. The thickness of the electroconductive layer may be about 5-50 μm.

In addition, an intermediate layer may be interposed between the substrate and the photosensitive layer or between the electroconductive layer and the photosensitive layer to enhance adhesion or to prevent charges from being injected from the substrate. Examples of the intermediate layer include, but are not limited to, an aluminum anodized layer; a resin-dispersed layer in which metal oxide powder such as titanium oxide or tin oxide is dispersed; and a resin layer such as polyvinyl alcohol, casein, ethylcellulose, gelatin, phenol resin, or polyamide. The thickness of the intermediate layer may be about 0.05-5 μm.

Also, each of the photosensitive layer, the electroconductive layer, and the intermediate layer may further comprise at least one additive selected from a plasticizer, a leveling agent, a dispersion stabilizing agent, an antioxidant, and an optical stabilizer, in addition to the binder resin.

Examples of the antioxidant include phenol compounds, sulfur compounds, phosphorus compounds, or amine compounds. Examples of the optical stabilizer include benzotriazole compounds, benzophenone compounds, or hindered amine compounds.

The electrophotographic photoreceptor according to an embodiment of the present invention may further comprise a surface protecting layer, if necessary.

Hereinafter, the present invention will be described in more detail with reference to the following examples. However, these examples are given for the purpose of illustration and are not intended to limit the scope of the invention.

### EXAMPLES

### Preparation Example 1

### Preparation of Compound (1)

The following is a description of the preparation of a naphthalenetetracarboxylic acid diimide having Formula (1) (Compound 1).

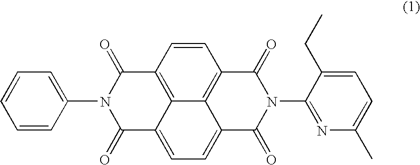

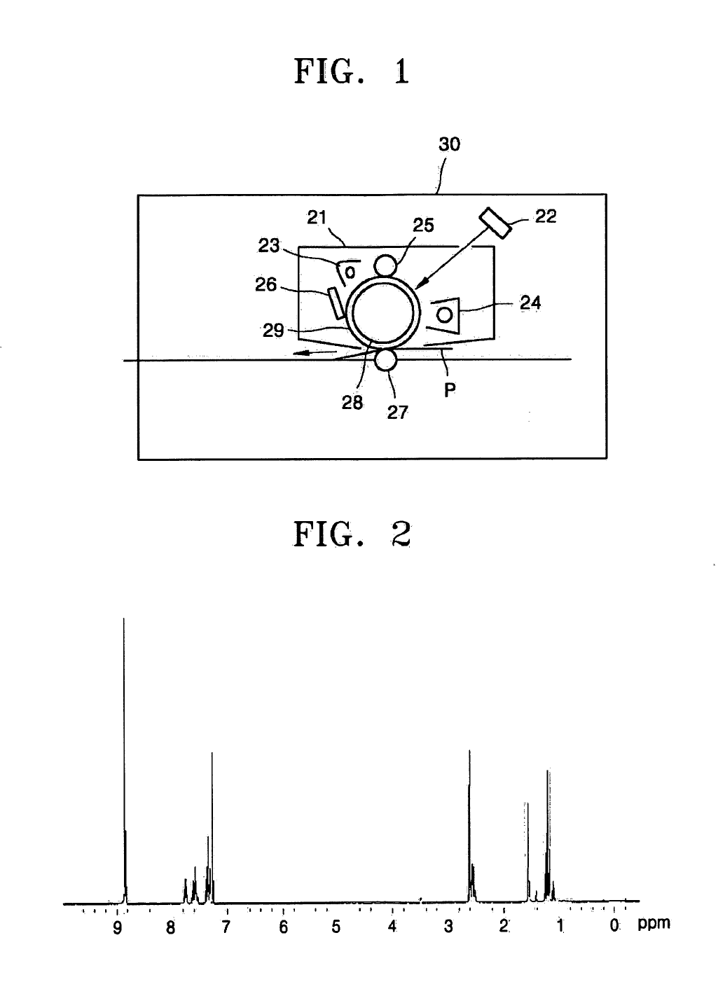

A 250 ml three neck flask equipped with a reflux condenser was purged with nitrogen, and then 13.4 g (0.05 mol) of 1,4,5,8-naphthalenetetracarboxylic acid dianhydride and 50 ml of N,N-dimethylformamide (DMF) were poured thereinto to be dissolved with stirring at room temperature, followed by raising the temperature of the mixture to reflux. Then, a mixture of 6.8 g (0.05 mol) of 2-amino-3-ethyl-6-methylpyridine, 4.7 g (0.05 mol) of aniline and 50 ml of DMF was slowly added dropwise, refluxed for 4 hours and cooled to room temperature. The reactant was added to 1000 ml of methanol to be precipitated and filtered. The filtered solid was recrystallized using a chloroform/ethanol solvent and vacuum dried to obtain 22.0 g of a crystal with a light yellow color (yield: 88%). The ¹H-NMR (300 MHz, CDCl₃ solvent) of the obtained (Compound 1) is shown in FIG. 2.

### Preparation Example 2

### Preparation of Compround (4)

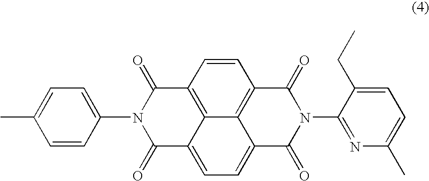

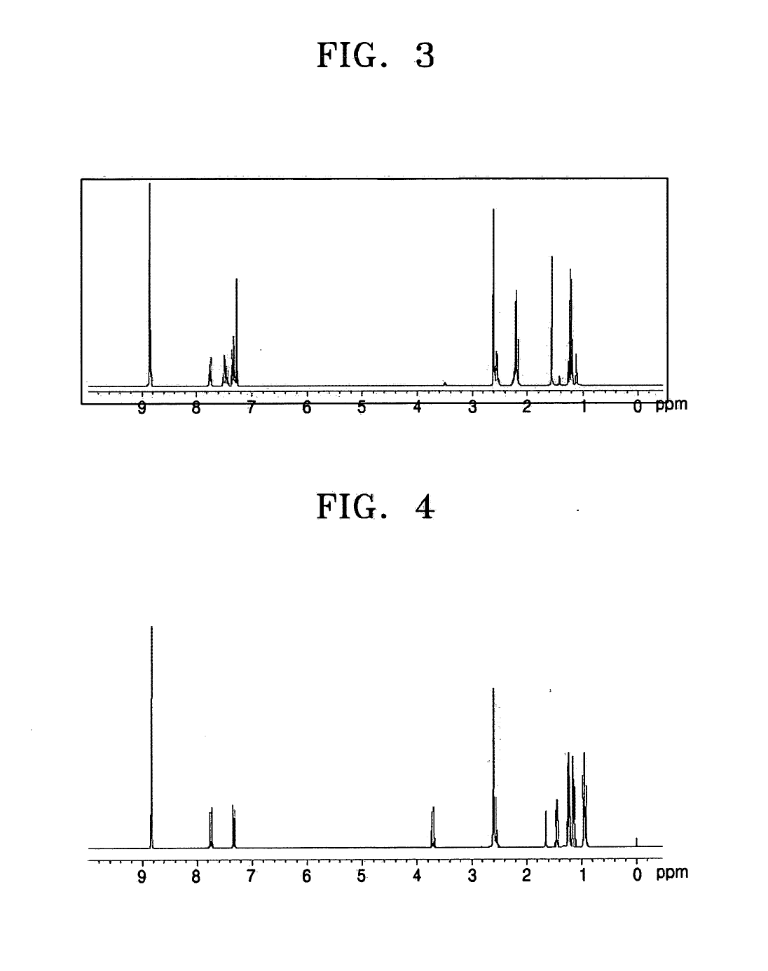

The naphthalenetetracarboxylic acid diimide (Compound (4)) was prepared in the same manner as in Preparation Example 1, except that 5.4 g (0.05 mol) of 4-methyl-aniline was used instead of aniline, yielding 21.1 g of a crystal with a light yellow color (yield: 89%). The ¹H-NMR (300 MHz, CDCl₃ solvent) of the obtained compound (4) is shown in FIG. 3.

### Preparation Example 3

### Preparation of Compound (5)

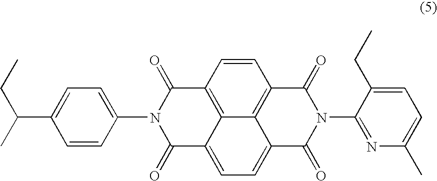

The naphthalenetetracarboxylic acid diimide (Compound (5)) was prepared in the same manner as in Preparation Example 1, except that 3.7 g (0.05 mol) of (R)-(−)-sec-butylamine was used instead of aniline, yielding 19.2 g of a crystal with a yellow color (yield: 87%). The ¹H-NMR (300MHz, CDCl₃ solvent) of the obtained compound (5) is shown in FIG. 4.

### Preparation Example 4

### Preparation of Compound (20)

The following is a description of the preparation of a naphthalenetetracarboxylic acid diimide (Compound (20)) to be used as ETM in Comparative Examples 1 and 2.

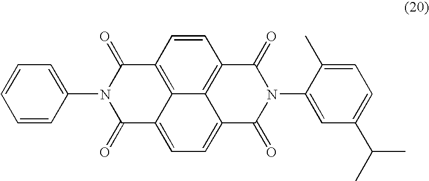

The naphthalenetetracarboxylic acid diimide (Compound 20) was prepared in the same manner as in Preparation Example 1, except that 6.6 g (0.05 mol) of 2-methyl-5-isopropylaniline was used instead of 2-amino-3-ethyl-6-methylpyridine, yielding 20.4 g of a crystal with a yellow color (yield: 86%).

### Preparation Example 5

### Preparation of Compound (30)

The following is a description of the preparation of a naphthalenetetracarboxylic acid diimide (Compound (30)) to be used as ETM in Comparative Examples 3 and 4.

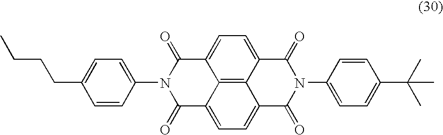

The naphthalenetetracarboxylic acid diimide (Compound (30)) was prepared in the same manner as in Preparation Example 1, except that 7.45 g (0.05 mol) of 4-n-butylaniline and 7.45 g (0.05 mol) of 4-t-butylaniline were used instead of aniline and 2-amino-3-ethyl-6-methylpyridine, respectively, yielding 22.2 g of a crystal with a yellow color (yield: 84%).

### Preparation Example 6

### Preparation of Compound (40)

The following is a description of the preparation of a naphthalenetetracarboxylic acid diimide (Compound (40)) to be used as ETM in Comparative Examples 5 and 6.

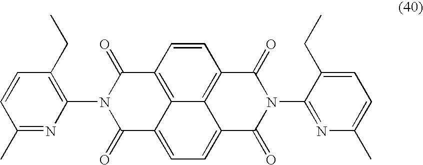

The naphthalenetetracarboxylic acid diimide (Compound (40)) was prepared in the same manner as in Preparation Example 1, except that only 13.6 g (0.1 mol) of 2-amino-3-ethyl-6-methylpyridine was used without using aniline, yielding 22.7 g of a crystal with a light orange color (yield: 90%).

### Preparation Example 7

### Preparation of ComPound (50)

The following is a description of the preparation of a naphthalenetetracarboxylic acid diimide (Compound (50)) to be used as ETM in Comparative Examples 7 and 8.

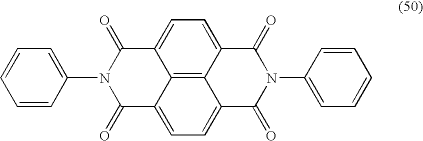

The naphthalenetetracarboxylic acid diimide (Compound (50)) was prepared in the same manner as in Preparation Example 1, except that only 9.4 g (0.1 mol) of aniline was used without using 2-amino-3-ethyl-6-methylpyridine, yielding 18.2 g of a crystal with a light orange color (yield: 87%).

### Example 1

4.5 parts by weight of the naphthalenetetracarboxylic acid diimide Compound (1) obtained in Preparation Example 1 as an ETM, 0.9 parts by weight of an X-type metal-free phthalocyanine Compound (6) (H2Pc) as a CGM, 9 parts by weight of an enaminestilbene-based Compound (7) as an HTM, 15.9 parts by weight of a binder resin Compound (8) (O-PET, available from KANEBO), 84 parts by weight of methylene chloride, and 36 parts by weight of 1,1,2-trichloroethane were sand milled for 2 hours and uniformly dispersed using ultrasonic waves.

The obtained solution was coated on an anodized aluminum drum (anodic oxide layer thickness: 5 μm) having a diameter of 3 cm by a ring coating method and dried at 110° C. for 1 hour to prepare an electrophotographic photoreceptor drum having a photosensitive layer having a thickness of about 14 to 15 μm.

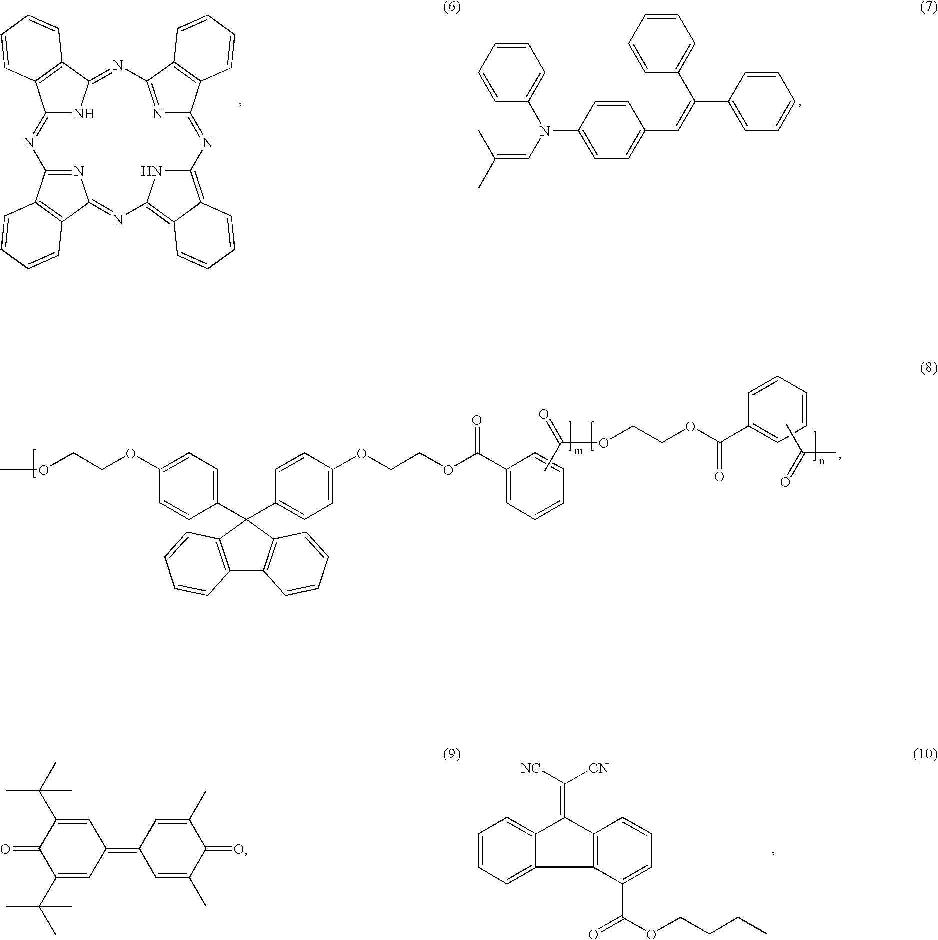

### Example 2

An electrophotographic photoreceptor drum was prepared in the same manner as in Example 1, except that the amount of the Compound (1) was adjusted to 4.05 parts by weight and 0.45 parts by weight of Compound (9) was further added as an electron acceptor (EA).

### Example 3

An electrophotographic photoreceptor drum was prepared in the same manner as in Example 1, except that 4.5 parts by weight of the Compound (4) was used instead of the Compound (1).

### Example 4

An electrophotographic photoreceptor drum was prepared in the same manner as in Example 1, except that the amount of the Compound (4) was adjusted to 4.05 parts by weight and 0.45 parts by weight of Compound (9) was further added as EA.

### Example 5

An electrophotographic photoreceptor drum was prepared in the same manner as in Example 1, except that 4.5 parts by weight of the Compound (5) was used instead of the Compound (1).

### Example 6

An electrophotographic photoreceptor drum was prepared in the same manner as in Example 5, except that the amount of the Compound (5) was adjusted to 4.05 parts by weight and 0.45 parts by weight of Compound (9) was further added as EA.

### Comparative Example 1

An electrophotographic photoreceptor drum was prepared in the same manner as in Example 1, except that 4.5 parts by weight of the Compound (20) was used instead of the Compound (1).

### Comparative Example 2

An electrophotographic photoreceptor drum was prepared in the same manner as in Comparative Example 1, except that the amount of the Compound (20) was adjusted to 4.05 parts by weight and 0.45 parts by weight of Compound (9) was further added as EA.

### Comparative Example 3

An electrophotographic photoreceptor drum was prepared in the same manner as in Example 1, except that 4.5 parts by weight of the Compound (30) was used instead of the Compound (1).

### Comparative Example 4

An electrophotographic photoreceptor drum was prepared in the same manner as in Comparative Example 3, except that the amount of the Compound (30) was adjusted to 4.05 parts by weight and 0.45 parts by weight of Compound (9) was further added as EA.

### Comparative Example 5

An electrophotographic photoreceptor drum was prepared in the same manner as in Example 1, except that 4.5 parts by weight of the Compound (40) was used instead of the Compound (1).

### Comparative Example 6

An electrophotographic photoreceptor drum was prepared in the same manner as in Comparative Example 5, except that the amount of the Compound (40) was adjusted to 4.05 parts by weight and 0.45 parts by weight of Compound (9) was further added as EA.

### Comparative Example 7

An electrophotographic photoreceptor drum was prepared in the same manner as in Example 1, except that 4.5 parts by weight of the Compound (50) was used instead of the Compound (1).

### Comparative Example 8

An electrophotographic photoreceptor drum was prepared in the same manner as in Comparative Example 7, except that the amount of the Compound (50) was adjusted to 4.05 parts by weight and 0.45 parts by weight of Compound (9) was further added as EA.

### Comparative Example 9

An electrophotographic photoreceptor drum was prepared in the same manner as in Example 1, except that 4.5 parts by weight of a dicyanofluorene Compound (10) was used instead of the Compound (1).

### Comparative Example 10

An electrophotographic photoreceptor drum was prepared in the same manner as in Comparative Example 9, except that the amount of the Compound (10) was adjusted to 4.05 parts by weight and 0.45 parts by weight of Compound (9) was further added as EA.

### Comparative Example 11

An electrophotographic photoreceptor drum was prepared in the same manner as in Example 1, except that the amount of an enaminestilbene-based Compound (7) was increased to 13.5 parts by weight without using the Compound (1).

### Comparative Example 12

An electrophotographic photoreceptor drum was prepared in the same manner as in Comparative Example 11, except that the amount of the Compound (7) was adjusted to 13.05 parts by weight and 0.45 parts by weight of Compound (9) was further added as EA.

### Solubility Test

1 g of a compound to be tested was poured into a test tube with stirring at room temperature and solvents listed in Table 1 were slowly added drop by drop. The amounts of the solvents required to completely dissolve 1 g of the tested compound were measured. Table 1 demonstrates the solubility test results.

TABLE 1

| | Acetone | Chloroform | Methylene | Tetrahydrofuran |
|---|---|---|---|---|
| | (g) | (g) | chloride (g) | (g) |
| Compound (1) | 130 | 5.3 | 6.5 | 5.8 |
| Compound (40) | 282 | 8.6 | 10.2 | 9.7 |

Referring to Table 1, the naphthalenetetracarboxylic acid diimide Compound (1) according to the present invention has approximately 1.8 to 2.2 times better solubility to the various organic solvents than the symmetric naphthalenetetracarboxylic acid diimide Compound (40).

### Long Period Charging Stability

Electrostatic properties of the respective electrophotographic photoreceptors prepared in the above Examples and Comparative Examples were measured using a scorotron-charging type drum photoreceptor evaluation apparatus manufactured by the Applicant of the present invention. The initial charge and exposure potentials and the charge and exposure potentials after 3,000 cycles were measured. The measured results are shown in Table 2. The drum photoreceptor evaluation apparatus has a drum diameter of 30 mm and a drum revolution speed of 5 ips (inch/second). The conditions of evaluation were as follows. A grid voltage (Vg)=1.0 kV, a wire current (lw)=300 uA, and laser supply unit (LSU) electrical power =0.9 mW.

In Table 2, the formation of crystals on the surface of the photoreceptor is indicated by O and an absence of crystal formation by X. In Table 2, O also refers to the presence of HTM and EA, respectively, and X refers to the absence of HTM and EA, respectively.

TABLE 2

| | | | | Surface | Vdᵢₙᵢₜᵢₐₗ | Vd₃₀₀₀ | Δ Vd | Voᵢₙᵢₜᵢₐₗ | Vo₃₀₀₀ | Δ Vo |
|---|---|---|---|---|---|---|---|---|---|---|
| | HTM | ETM | EA | Crystallization | (V) | (V) | (V) | (V) | (V) | (V) |
| Example 1 | ◯ | Compound | X | X | 77 | 83 | 6 | 875 | 875 | 0 |
| | | (1) | | | | | | | | |
| Example 2 | ◯ | Compound | ◯ | X | 62 | 67 | 5 | 882 | 882 | 0 |
| | | (1) | | | | | | | | |
| Example 3 | ◯ | Compound | X | X | 72 | 80 | 8 | 880 | 880 | 0 |
| | | (4) | | | | | | | | |
| Example 4 | ◯ | Compound | ◯ | X | 65 | 68 | 3 | 892 | 892 | 0 |
| | | (4) | | | | | | | | |
| Example 5 | ◯ | Compound | X | X | 80 | 88 | 8 | 866 | 866 | 0 |
| | | (5) | | | | | | | | |
| Example 6 | ◯ | Compound | ◯ | X | 68 | 72 | 4 | 875 | 875 | 0 |
| | | (5) | | | | | | | | |
| Comparative | ◯ | Compound | X | X | 85 | 90 | 5 | 850 | 809 | 41 |
| Example 1 | | (20) | | | | | | | | |
| Comparative | ◯ | Compound | ◯ | X | 84 | 90 | 6 | 866 | 850 | 16 |
| Example 2 | | (20) | | | | | | | | |
| Comparative | ◯ | Compound | X | X | 87 | 91 | 4 | 864 | 822 | 42 |
| Example 3 | | (30) | | | | | | | | |
| Comparative | ◯ | Compound | ◯ | X | 88 | 92 | 4 | 870 | 852 | 18 |
| Example 4 | | (30) | | | | | | | | |
| Comparative | ◯ | Compound | X | X | 87 | 91 | 4 | 897 | 810 | 87 |
| Example 5 | | (40) | | | | | | | | |
| Comparative | ◯ | Compound | ◯ | X | 88 | 92 | 4 | 900 | 887 | 13 |
| Example 6 | | (40) | | | | | | | | |
| Comparative | ◯ | Compound | X | ◯ | 195 | 200 | 5 | 827 | 644 | 183 |
| Example 7 | | (50) | | | | | | | | |
| Comparative | ◯ | Compound | ◯ | ◯ | 155 | 158 | 3 | 808 | 627 | 181 |
| Example 8 | | (50) | | | | | | | | |
| Comparative | ◯ | Compound | X | X | 110 | 132 | 22 | 825 | 645 | 180 |
| Example 9 | | (10) | | | | | | | | |
| Comparative | ◯ | Compound | ◯ | X | 100 | 107 | 7 | 875 | 809 | 66 |
| Example 10 | | (10) | | | | | | | | |
| Comparative | ◯ | — | X | X | 150 | 165 | 15 | 750 | 565 | 185 |
| Example 11 | | | | | | | | | | |
| Comparative | ◯ | — | ◯ | X | 134 | 140 | 6 | 723 | 597 | 126 |
| Example 12 | | | | | | | | | | |

In Table 2, V_{o initial} denotes an initial charge potential, V_{d initial} denotes an initial exposure potential, V_{o 3000} denotes a charge potential after 3000 cycles, and V_{d 3000} denotes an exposure potential after 3000 cycles. ΔV_{d} refers to an increase in the exposure potential after 3000 cycles, i.e., ΔV_{d}=V_{d 3000}−V_{d initial}.

ΔVₒ refers to a decrease in the charge potential after 3000 cycles, i.e., ΔVₒ=V_{o initial}−V_{o 3000}.

ΔVo and ΔVd indicate changes in the surface potentials of the electrophotographic photoreceptor after several thousand cycles. When the photoreceptor having high ΔVo and ΔVd values is used for image forming, image qualities deteriorate with repetition of cycles.

Referring to Table 2, surprisingly, the photoreceptor drums prepared in Examples 1 through 6 using the pyridine-substituted asymmetric naphthalenetetracarboxylic acid diimide compounds according to the present invention (Compounds (1), (4) and (5)) showed no reduction in charge potential after 3000 cycles (ΔVo=O).

In contrast, the photoreceptor drums prepared in Comparative Examples 1 through 4 using the phenyl-substituted asymmetric naphthalenetetracarboxylic acid diimide compounds (Compounds (20) and (30)) as an ETM, as disclosed in U.S. Pat. No. 4,992,349, undesirably showed a considerable reduction in charge potential after 3000 cycles (ΔVo=16˜42 V). Comparative Examples 2 and 4, in which electron acceptors were used in the production of the photoreceptor, ΔVₒ=16-18 V, was less than the decrease in the charge potential ΔVₒ=41-42 V of Comparative Examples 1 and 3, where the electron acceptors were not used in the production of the photoreceptor. Thus, it was confirmed that when a phenyl-substituted asymmetric naphthalenetetracarboxylic acid diimide compound (Compounds (20) and (30)) is used as an ETM, a decrease in the charge potential can be reduced by using the electron acceptor in the production of the photoreceptor. However, in Examples 1 through 6, the charge potential showed no decrease(ΔVo=O), i.e., an electrostatic property showed good stability in charge potential even without using the electron acceptor. That is, the charge potential even after several thousand cycles may not be greatly decreased in the electrophotographic photoreceptor using the pyridine-substituted asymmetric naphthalenetetracarboxylic acid diimide compound according to the present invention as the ETM. Therefore, the lifetime of the electrophotographic photoreceptor can be extended. Also, the image quality can be maintained after use for an extended time.

A comparison of charge potential stability was made for Examples 1 through 6 and Comparative Examples 5 and 6 in which pyridine-substituted naphthalenetetracarboxylic acid diimide compounds were used as an ETM. The comparison results showed that the electrophotographic photoreceptor drums prepared in Examples 1 through 6 using the asymmetric Compounds (1), (4) or (5) exhibited much better charge potential stability than the electrophotographic photoreceptor drums prepared in Comparative Examples 5 and 6 using the symmetric compound (40).

The electrophotographic photoreceptor drums prepared in Comparative Examples 7 through 12 had relatively large values of ΔVo and ΔVd, and exhibited poor electrostatic properties. Particularly, the electrophotographic photoreceptor drums prepared in Comparative Examples 7 and 8 in which the phenyl-substituted symmetric naphthalenetetracarboxylic acid diimide compounds were used as an ETM exhibited surfaces of the photoreceptors that were crystallized in the production of the electrophotographic photoreceptor drums due to low solubility in an organic solvent and low compatibility with a polymer binder resin. Thus, the electrostatic property was remarkably deteriorated compared to the Examples 1 through 6.

As described above, the pyridine-substituted asymmetric naphthalenetetracarboxylic acid diimide compound according to the present invention has increased solubility in organic solvents, improved compatibility with binder resins and enhanced resistance for nitrogen oxides. Thus, an electrophotographic photoreceptor containing the asymmetric naphthalenetetracarboxylic acid diimide compound according to the present invention can maintain a constant surface potential and durability after being repeatedly used for an extended time. Thus, when the electrophotographic photoreceptor according to the present invention can provide a high image quality for an extended time.

Although a few preferred embodiments of the present invention have been shown and described, it would be appreciated by those skilled in the art that changes may be made in this embodiment without departing from the principles and spirit of the invention, the scope of which is defined in the claims and their equivalents.

What is claimed is:

1. An electrophotographic photoreceptor comprising:
an electrically conductive substrate; and
a photosensitive layer formed on the electrically conductive substrate,
wherein the photosensitive layer comprises a pyridine-substituted asymmetric naphthalenetetracarboxylic acid diimide derivative represented by Formula (1): 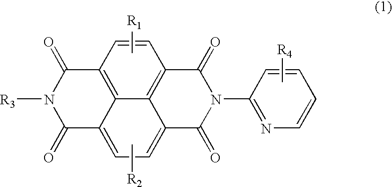
wherein R₁, R₂ R₃ and R₄ are independently selected from the group consisting of a substituted or unsubstituted C₁-C₂₀ alkyl group, a substituted or unsubstituted C₁-C₂₀ alkoxy group, a substituted or unsubstituted C₆-C₃₀ aryl group, a substituted or unsubstituted C₇-C₃₀ aralkyl group, a substituted or unsubstituted C₃-C₃₀ heterocyclic group, and a halogen atom.

2. The electrophotographic photoreceptor of claim 1, further comprising an intermediate layer between the electrically conductive substrate and the photosensitive layer.

3. An electrophotographic imaging apparatus comprising an electrophotographic photoreceptor, wherein the electrophotographic photoreceptor comprises:
an electrically conductive substrate; and
a photosensitive layer formed on the electrically conductive substrate,
wherein the photosensitive layer comprises a pyridine-substituted asymmetric naphthalenetetracarboxylic acid diimide derivative represented by Formula (1): 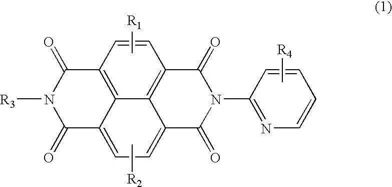
wherein R₁, R₂ R₃ and R₄ are independently selected from the group consisting of a substituted or unsubstituted C₁-C₂₀ alkyl group, a substituted or unsubstituted C₁-C₂₀ alkoxy group, a substituted or unsubstituted C₆-C₃₀ aryl group, a substituted or unsubstituted C₇-C₃₀ aralkyl group, a substituted or unsubstituted C₃-C₃₀ heterocyclic group, and a halogen atom.

4. The electrophotographic imaging apparatus of claim 3, further comprising an intermediate layer between the electrically conductive substrate and the photosensitive layer.

5. An electrophotographic cartridge comprising:
an electrophotographic photoreceptor comprising an electrically conductive substrate, and a photosensitive layer formed on the electrically conductive substrate, wherein the photosensitive layer comprises a pyridine-substituted asymmetric naphthalenetetracarboxylic acid diimide derivative represented by Formula (1): 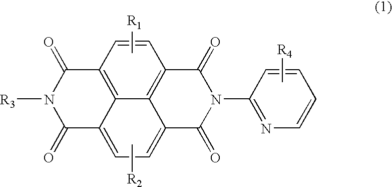
wherein R₁, R₂ R₃ and R₄ are independently selected from the group consisting of a substituted or unsubstituted C₁-C₂₀ alkyl group, a substituted or unsubstituted C₁-C₂₀ alkoxy group, a substituted or unsubstituted C₆-C₃₀ aryl group, a substituted or unsubstituted C₇-C₃₀ aralkyl group, a substituted or unsubstituted C₃-C₃₀ heterocyclic group, and a halogen atom; and
at least one selected from the group consisting of a charging device that charges the electrophotographic photoreceptor, a developing device that develops an electrostatic latent image formed on the electrophotographic photoreceptor, and a cleaning device that cleans a surface of the electrophotographic photoreceptor, the electrophotographic cartridge being attachable to or detachable from an imaging apparatus.

6. The electrophotographic cartridge of claim 5, further comprising an intermediate layer between the electrically conductive substrate and the photosensitive layer.

7. An electrophotographic imaging apparatus comprising:
a photoreceptor unit comprising an electrically conductive substrate, and a photosensitive layer formed on the electrically conductive substrate, wherein the photosensitive layer comprises a pyridine-substituted asymmetric naphthalenetetracarboxylic acid diimide derivative represented by Formula (1): 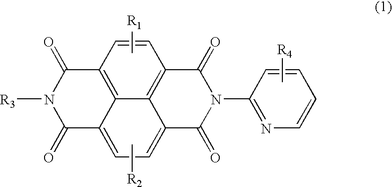
wherein R₁, R₂ R₃ and R₄ are independently selected from the group consisting of a substituted or unsubstituted C₁-C₂₀ alkyl group, a substituted or unsubstituted C₁-C₂₀ alkoxy group, a substituted or unsubstituted C₆-C₃₀ aryl group, a substituted or unsubstituted C₇-C₃₀ aralkyl group, a substituted or unsubstituted C₃-C₃₀ heterocyclic group, and a halogen atom;
a charging device that charges the photoreceptor unit;
an imagewise light irradiating device that irradiates the charged photoreceptor unit with imagewise light to form an electrostatic latent image on the photoreceptor unit;
a developing unit that develops the electrostatic latent image with a toner to form a toner image on the photoreceptor unit; and
a transfer unit that transfers the toner image onto a receiving material.

8. The electrophotographic imaging apparatus of claim 7, further comprising an intermediate layer between the electrically conductive substrate and the photosensitive layer.

9. A pyridine-substituted asymmetric naphthalenetetracarboxylic acid diimide derivative represented by Formula (1): 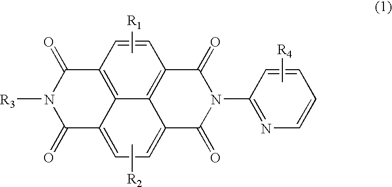
wherein R₁, R₂ R₃ and R₄ are independently selected from the group consisting of a substituted or unsubstituted C₁-C₂₀ alkyl group, a substituted or unsubstituted C₁-C₂₀ alkoxy group, a substituted or unsubstituted C₆-C₃₀ aryl group, a substituted or unsubstituted C₇-C₃₀ aralkyl group, a substituted or unsubstituted C₃-C₃₀ heterocyclic group, and a halogen atom.

10. The pyridine-substituted asymmetric naphthalenetetracarboxylic acid diimide derivative of claim 9, wherein said derivative is selected from the group consisting of [chemical structure]
